(19) United States
(12) Patent Application Publication
(10) Pub. No.: US 2026/0210821 A1
(43) Pub. Date: Jul. 23, 2026

(54) METHOD FOR ESTABLISHING AND PREDICTING ENERGY-TIME DENSITY MAP MODEL FOR EVALUATING ROCK ENERGY CONSUMPTION

(71) Applicant: SHENYANG UNIVERSITY OF TECHNOLOGY, Shenyang (CN)

(72) Inventors: Ding DENG, Shenyang (CN); Lianjun GUO, Shenyang (CN)

(21) Appl. No.: 19/454,977

(22) Filed: Jan. 21, 2026

(30) Foreign Application Priority Data
Jan. 23, 2025 (CN) 202510107428.4

(51) Int. Cl. G01N 3/30; G01N 1/02; G01N 33/24

(52) U.S. Cl. CPC G01N 3/30; G01N 1/02; G01N 33/24; G01N 2001/021; G01N 2203/001; G01N 2203/0067; G01N 2203/0098; G01N 2203/0252

(57) ABSTRACT

A method for establishing and predicting an energy-time density map model for evaluating rock energy consumption is provided. The method includes the following steps: selecting rock samples and conducting Hopkinson experiments to obtain stress and strain signals; processing the obtained stress-strain signals by using a deep learning denoising model to obtain purified stress-strain signals; calculating dynamic strength data and energy-time density based on the purified stress-strain signals; for various rocks, constructing a two-dimensional energy-time density map model by using a K-means clustering algorithm and kernel density estimation; for a single rock, constructing an intuitive continuous two-dimensional energy-time density map by using an interpolation model of machine learning; the two-dimensional energy-time density map may predict the variation trend of energy-time density, thereby analyzing the energy dissipation situation.

### CROSS-REFERENCE TO RELATED APPLICATIONS

This application claims priority to Chinese Patent Application No. 202510107428.4, filed on Jan. 23, 2025, the contents of which are hereby incorporated by reference.

### TECHNICAL FIELD

The present disclosure relates to the technical field of rock mechanics, and in particular to a method for establishing and predicting an energy-time density map model for evaluating rock energy consumption.

### BACKGROUND

In the fields of rock mechanics and energy, it is often necessary to extract required rock materials. Rock materials are subjected to various types of external loads that induce fragmentation. However, the fastest mining method currently is still to peel and break rocks through dynamic loads such as blasting, and then the extracted rocks are further refined and processed. Studying the energy dissipation characteristics of rocks under external dynamic loads is of great significance for understanding their mechanical behavior, fracture mechanism, and energy utilization. Conventional energy evaluation methods only discuss dissipated energy and are relatively rough, making it difficult to accurately describe the temporal and spatial variation laws during energy consumption. Energy-time density may take both time and spatial factors into account, and may evaluate the energy relationship of rocks under various load conditions, which may more comprehensively reflect the energy relationship of rocks under various load conditions. The existing technology lacks data quality control and pattern recognition means in the process of energy-time density analysis. Complex test signals are often accompanied by a lot of noise, and the time series of rock dissipation process often has nonlinear characteristics and potential patterns that are difficult to accurately capture through traditional means. With the development of machine learning and deep learning technologies, it has become possible to realize data preprocessing, feature extraction and pattern prediction through intelligent means. At the same time, there is a lack of energy-time density ranges for various rocks. Based on the energy-time density index, establishing a map model as an evaluation method for rock energy consumption by using related technologies such as machine learning and deep learning is of great scientific significance and application value for rock energy dissipation research.

### SUMMARY

The purpose of the present disclosure is to provide a method for establishing and predicting an energy-time density map model for evaluating rock energy consumption, aiming at solving the problems existing in the traditional energy evaluation methods that only discuss dissipated energy and are relatively rough, making it difficult to accurately describe the temporal and spatial variation laws during energy consumption.

To achieve the above purpose, according to a first aspect of the present disclosure, a method for establishing an energy-time density map model for evaluating rock energy consumption is provided. The method for establishing the energy-time density map model includes the following steps:
selecting rock samples, and conducting Hopkinson experiments to obtain stress and strain signals;
processing an obtained stress-strain signals by using a deep learning denoising model to obtain purified stress-strain signals;
calculating dynamic strength data and energy-time density based on the purified stress-strain signals; and
constructing a two-dimensional energy-time density map model based on the dynamic strength data and the energy-time density of rocks, and by using a K-means clustering algorithm, kernel density estimation or an interpolation model of machine learning.

In an embodiment, the method of selecting rock samples and conducting Hopkinson experiments to obtain stress and strain signals specifically includes:
collecting and processing multiple types of the rock samples into specimen sizes required for the Hopkinson experiments; and
conducting the Hopkinson experiments on the rock samples to be tested, performing five experiments for each type of the rock samples, and collecting stress and strain signals of the rocks under impact loads.

In an embodiment, the method of processing the obtained stress-strain signals by using a deep learning denoising model specifically includes:
inputting collected stress and strain signal data into the deep learning denoising model, and automatically filtering high-frequency noise in data through a denoising autoencoder (DAE) to obtain the purified stress-strain signals.

In an embodiment, the specific steps for obtaining the dynamic strength data include:
a calculation method of calculating the dynamic strength data of various rocks based on the purified stress-strain signals and combined with one-dimensional stress wave theory of elastic rods is as follows:

$$\{ \begin{matrix}σ ( t ) = \frac{AE}{2 {A}_{s}} [ {ε}_{I} ( t ) + {ε}_{R} ( t ) + {ε}_{T} ( t ) ] \\ ε ( t ) = \to \frac{C}{{l}_{s}} {\int }_{0}^{t} [ {ε}_{I} ( t ) - {E}_{R} ( r ) - {ε}_{T} ( i ) dt \\ \dot{ε} ( t ) = \frac{C}{{l}_{s}} [ {ε}_{I} ( t ) - {ε}_{R} ( t ) - {ε}_{T} ( t ) ]\end{matrix} ;$$

where A, E, and C are a cross-sectional area, elastic modulus, and longitudinal wave velocity of a pressure bar, respectively; Aₛ and lₛ are a cross-sectional area and a length of a specimen, respectively; and ε_{I}(t), ε_{R}(t), and ε_{T}(t) are incident strain, reflected strain, and transmitted strain of the pressure bar;

In an embodiment, the specific steps for obtaining the energy-time density include:
calculating the energy-time density under a same load condition by using a three-wave method based on the purified stress-strain signals;
where calculation methods of each part of energy are as follows:

$${W}_{i} = \frac{{A}_{0}}{{ρ}_{0} {C}_{0}} {\int }_{0}^{t} {σ}_{i}^{2} ( t ) dt ;$$

$${W}_{r} = \frac{{A}_{0}}{{ρ}_{0} {C}_{0}} {\int }_{0}^{t} {σ}_{r}^{2} ( t ) dt ;$$

$${W}_{t} = \frac{{A}_{0}}{{ρ}_{0} {C}_{0}} {\int }_{0}^{t} {σ}_{t}^{2} ( t ) dt ; and$$

$${W}_{d} = {W}_{i} - {W}_{r} - {W}_{t} ;$$

where Wᵢ, Wᵣ, Wₜ, and W_{d} are incident energy, reflected energy, transmitted energy, and absorbed energy, respectively; σᵢ, σᵣ, and σₜ are incident stress, reflected stress, and transmitted stress, respectively; A₀ is a cross-sectional area of a rod; C₀ is elastic wave velocity in the pressure bar; ρ₀ is density of an elastic rod;
the energy consumption density is defined as dissipated energy per unit volume

$${U}_{d} = \frac{{W}_{d}}{V} ;$$

where U_{d} is the energy consumption density, W_{d} is the absorbed energy, and V is a volume; and
the energy-time density of the rocks is calculated according to the energy consumption density; the energy-time density reflects dissipated energy of the rock per unit volume per unit time, and a calculation formula is as follows:

$${E}_{VT} = \frac{{U}_{d}}{T} ;$$

where E_{VT} is the energy-time density, U_{d} is the energy consumption density, and T is action time of a reflected wave.

In an embodiment, a method of constructing the two-dimensional energy-time density map model based on the dynamic strength data and the energy-time density of the rocks, and by using the K-means clustering algorithm, the kernel density estimation or the interpolation model of the machine learning includes:
(a) based on energy-time density and dynamic strength data of the rocks under a same load, taking dynamic strength as an abscissa and a peak value of the energy-time density as an ordinate, drawing a basic energy-time density distribution scatter diagram, and calculating a category and density probability of each scatter based on the K-means clustering algorithm and the kernel density estimation to generate the two-dimensional energy-time density map model;
calculating a position of each energy-time density and each dynamic strength, and performing cluster analysis to obtain the density probability of each cluster; assuming that a data point set is D={(x₁,y₁), (x₂,y₂), . . . , (xₙ,yₙ)}, where x is the dynamic strength and y is the peak value of the energy-time density, and based on the K-means clustering algorithm, an objective function of a clustering process may be expressed as:

$$J = {{\sum }_{k = 1}}^{K} {{\sum }_{i = 1}}^{n} {‖ ( {x}_{i} , {y}_{i} ) - {c}_{k} ‖}^{2} ;$$

where J is the objective function, K is a number of clusters, cₖ is a center of a k-th cluster, and ∥(xᵢ, yᵢ)−cₖ∥ is a Euclidean distance between data points and a cluster center;
using the kernel density estimation to estimate the probability density of the data points and generating a two-dimensional probability density map:

$$f ( x , y ) = \frac{1}{{nh}_{x} {h}_{y}} {\sum }_{i = 1}^{n} K ( \frac{x - {x}_{i}}{{h}_{x}} , \frac{y - {y}_{i}}{{h}_{y}} ) ;$$

where f(x,y) is a probability density function, n is a number of the data points, hₓ and h_{y} are bandwidth parameters, and K is a kernel function, usually a Gaussian kernel;
(b) based on energy-time density and dynamic strength data of each of the rocks under different loads, interpolating and extrapolating energy-time density data under different strain rates, and using the interpolation model of the machine learning to construct a continuous and smooth spatial distribution map of the energy-time density to obtain a two-dimensional energy-time density map distribution model;
using the interpolation model of the machine learning to interpolate the energy-time density under different strain rates:

$$\hat{y} = f ( x , θ ) = W \cdot x + b ;$$

where ŷ is a predicted energy-time density value, f(x,θ) is an output of an multiplayer perceptron (MLP) model, W is a weight matrix, b is a bias term, and x is an input feature.

According to a second aspect of the present disclosure, a method for predicting an energy-time density map model for evaluating rock energy consumption is also provided. The prediction method is to perform trend prediction based on the two-dimensional energy-time density map model to obtain an energy-time density prediction result.

In an embodiment, the method of performing the trend prediction based on the two-dimensional energy-time density map model to obtain the energy-time density prediction result includes:
extracting energy-time density with a maximum or minimum eigenvalue and a rising trend or a falling trend of the energy-time density from the two-dimensional energy-time density map model; and
generating a trend curve based on extracted eigenvalues, and predicting corresponding energy-time density result by inputting new dynamic strength data based on a generated trend curve to provide a reference for engineering decisions.

Compared with the prior art, the beneficial effects of the present disclosure are as follows.

Improved accuracy: By introducing a deep learning denoising model to intelligently denoise experimental data and combining with deep learning feature extraction technology, the accuracy and reliability of energy-time density calculation are effectively improved. This method not only reduces the impact of noise on the results but also enhances the ability to capture key features, providing high-quality data support for subsequent analysis.

Enhanced visualization: The analysis method based on machine learning provides an intuitive and in-depth visualization for the energy-time density map.

Wide applicability and dynamic analysis: It is applicable to various types of rocks and different experimental loading conditions. Through the prediction model, the energy dissipation under various conditions may be analyzed.

Prediction and decision support: The prediction ability of deep learning is used to predict energy dissipation, providing a scientific basis for the optimization of engineering blasting schemes, rock fracture control, and energy utilization decisions.

Based on the implementation methods provided in the above aspects, the present disclosure may further combine to provide more implementation methods.

### BRIEF DESCRIPTION OF THE DRAWINGS

To more clearly illustrate the technical schemes in the embodiments of the present disclosure, the drawings required in the description of the embodiments will be briefly introduced below. Apparently, the drawings in the following description are only some embodiments of the present disclosure. For those of ordinary skill in the art, other drawings may be obtained according to these drawings without creative work.

FIG. 1 is a flow chart of calculating and compiling the energy-time density map for evaluating rock energy consumption according to the present disclosure.

FIG. 2A is the original diagram of stress-strain data.

FIG. 2B is the diagram after DAE smoothing.

FIG. 3A is the two-dimensional map model of typical rocks.

FIG. 3B is the two-dimensional map model of typical loads.

FIG. 4 is a flow chart of prediction effect and decision analysis.

FIG. 5A is the two-dimensional map model of typical rocks in metal mines.

FIG. 5B is the two-dimensional map model of typical loads in metal mines.

FIG. 6A is the two-dimensional map model of typical rocks in limestone mines.

FIG. 6B is the two-dimensional map model of typical loads in limestone mines.

FIG. 7 is a flow chart of a method of selecting the rock samples and conducting the Hopkinson experiments to obtain the stress and the strain signals.

FIG. 8 is a flow chart of a method of processing the obtained stress-strain signals by using the deep learning denoising model.

FIG. 9 is a flow chart of a method of performing the trend prediction based on the two-dimensional energy-time density map model to obtain the energy-time density prediction result.

### DETAILED DESCRIPTION OF THE EMBODIMENTS

To enable those skilled in the art to better understand the schemes of the present disclosure, the technical schemes in the embodiments of the present disclosure will be clearly and completely described below in conjunction with the drawings in the embodiments of the present disclosure. Apparently, the described embodiments are only part of the embodiments of the present disclosure, not all of them. Based on the embodiments of the present disclosure, all other embodiments obtained by those of ordinary skill in the art without creative work shall fall within the protection scope of the present disclosure. Unless otherwise specified, the technical means used in the embodiments are conventional means well known to those skilled in the art.

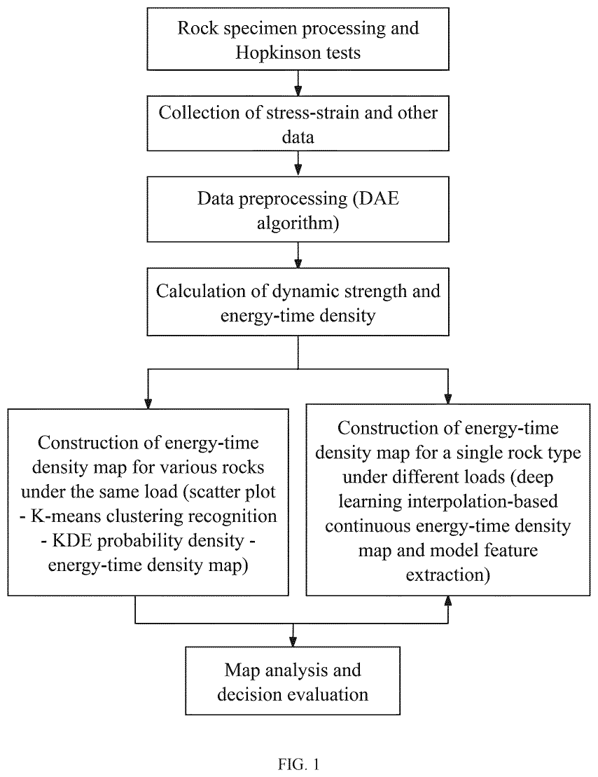

An embodiment of the present disclosure provides a method for establishing an energy-time density map model for evaluating rock energy consumption. As shown in FIG. 1, FIG. 1 is a flow chart of a method for establishing an energy-time density map model for evaluating rock energy consumption provided by an embodiment of the present disclosure. The method for establishing the energy-time density map model includes the following steps:
selecting rock samples and conducting Split Hopkinson Pressure Bar (SHPB (Hopkinson)) experiments to obtain stress and strain signals;
processing an obtained stress-strain signals by using a deep learning denoising model to obtain purified stress-strain signals;
calculating dynamic strength data and energy-time density based on the purified stress-strain signals; and
constructing a two-dimensional energy-time density map model based on the dynamic strength data and the energy-time density of rocks, and by using a K-means clustering algorithm, kernel density estimation (KDE) or an interpolation model of machine learning.

Based on the establishment of an energy-time density map model for evaluating rock energy consumption provided by an embodiment of the present disclosure, the stress-strain signals obtained by SHPB experiments are purified through a deep learning denoising model, which improves the data quality and the accuracy of subsequent calculations. By using the purified signals, the dynamic strength and energy-time density of rocks are accurately calculated, and an intuitive two-dimensional energy-time density map is constructed by combining algorithms such as K-means clustering and KDE probability density estimation.

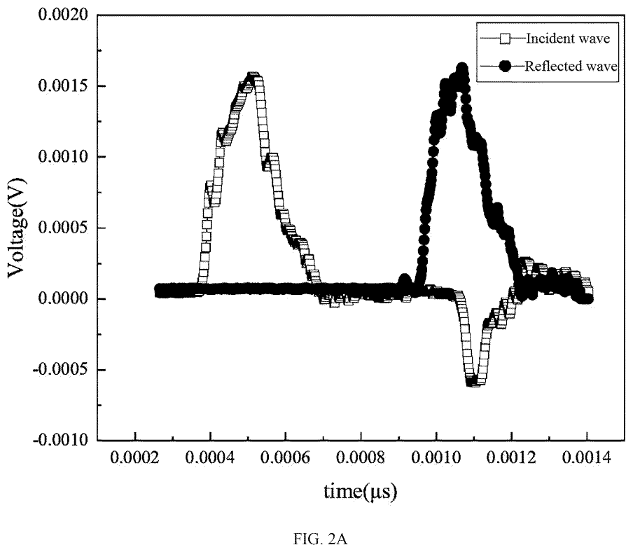

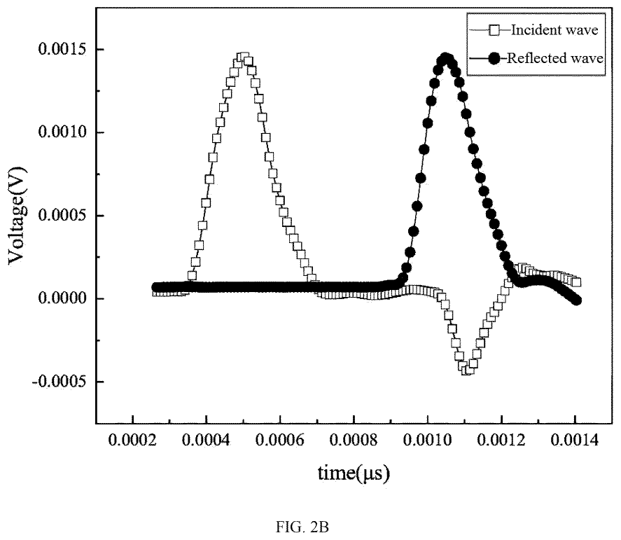

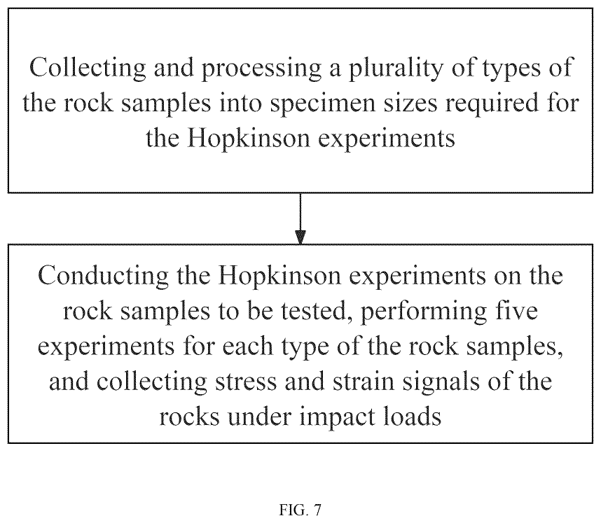

In one embodiment, as shown in FIG. 7, a method of selecting the rock samples and conducting the Hopkinson experiments to obtain the stress and the strain signals includes:
collecting and processing multiple types of the rock samples into specimen sizes required for the Hopkinson experiments; and
conducting the Hopkinson experiments on the rock samples to be tested, performing five experiments for each type of the rock samples, and collecting stress and strain signals of the rocks under impact loads, as shown in FIG. 2A.

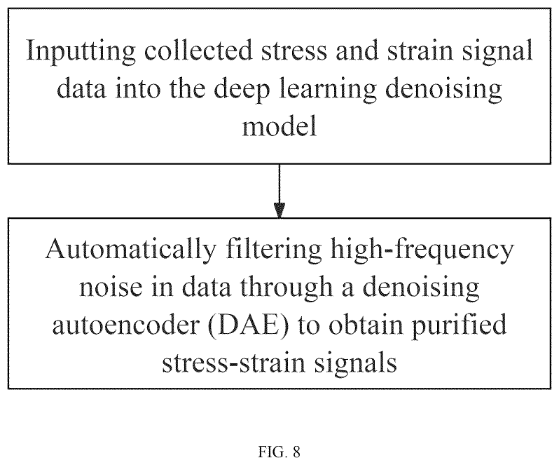

In one embodiment, as shown in FIG. 8, a method of processing the obtained stress-strain signals by using the deep learning denoising model includes:
inputting collected stress and strain signal data into the deep learning denoising model, and automatically filtering high-frequency noise in data through a denoising autoencoder (DAE) to obtain the purified stress-strain signals, as shown in FIG. 2B.

In one embodiment, specific steps for obtaining the dynamic strength data include:
calculating the dynamic strength data of various rocks based on the purified stress-strain signals and combined with one-dimensional stress wave theory of elastic rods, a calculation method is as follows:

$$\{ \begin{matrix}σ ( t ) = \frac{AE}{2 {A}_{s}} [ {ε}_{I} ( t ) + {ε}_{R} ( t ) + {ε}_{T} ( t ) ] \\ ε ( t ) = \to \frac{C}{{l}_{s}} {\int }_{0}^{t} [ {ε}_{I} ( t ) - {E}_{R} ( r ) - {ε}_{T} ( i ) dt \\ \dot{ε} ( t ) = \frac{C}{{l}_{s}} [ {ε}_{I} ( t ) - {ε}_{R} ( t ) - {ε}_{T} ( t ) ]\end{matrix} ;$$

where A, E, and C are a cross-sectional area, elastic modulus, and longitudinal wave velocity of a pressure bar, respectively; Aₛ and lₛ are a cross-sectional area and a length of a specimen, respectively; and ε_{I}(t), ε_{R}(t), and ε_{T}(t) are incident strain, reflected strain, and transmitted strain of the pressure bar.

In one embodiment, the steps for obtaining the energy-time density include:
calculating the energy-time density under a same load condition by using a three-wave method based on the purified stress-strain signals;
where calculation methods of each part of energy are as follows:

$${W}_{i} = \frac{{A}_{0}}{{ρ}_{0} {C}_{0}} {\int }_{0}^{t} {σ}_{i}^{2} ( t ) dt ;$$

$${W}_{r} = \frac{{A}_{0}}{{ρ}_{0} {C}_{0}} {\int }_{0}^{t} {σ}_{r}^{2} ( t ) dt ;$$

$${W}_{t} = \frac{{A}_{0}}{{ρ}_{0} {C}_{0}} {\int }_{0}^{t} {σ}_{t}^{2} ( t ) dt ; and$$

$${W}_{d} = {W}_{i} - {W}_{r} - {W}_{t} ;$$

where Wᵢ, Wᵣ, Wₜ, and W_{d} are incident energy, reflected energy, transmitted energy, and absorbed energy, respectively; σᵢ, σᵣ, and σₜ are incident stress, reflected stress, and transmitted stress, respectively; A₀ is a cross-sectional area of a rod; C₀ is elastic wave velocity in the pressure bar; ρ₀ is density of an elastic rod;
the energy consumption density is defined as dissipated energy per unit volume:

$${U}_{d} = \frac{{W}_{d}}{V} ;$$

where U_{d} is the energy consumption density, W_{d} is the absorbed energy, and V is a volume; and
the energy-time density of the rocks is calculated according to the energy consumption density; the energy-time density reflects dissipated energy of the rock per unit volume per unit time, and a calculation formula is as follows:

$${E}_{VT} = \frac{{U}_{d}}{T} ;$$

where E_{VT} is the energy-time density, U_{d} is the energy consumption density, and T is action time of a reflected wave.

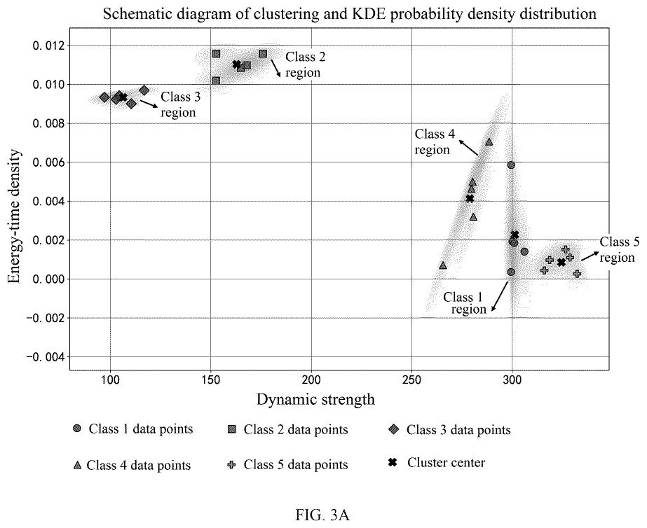

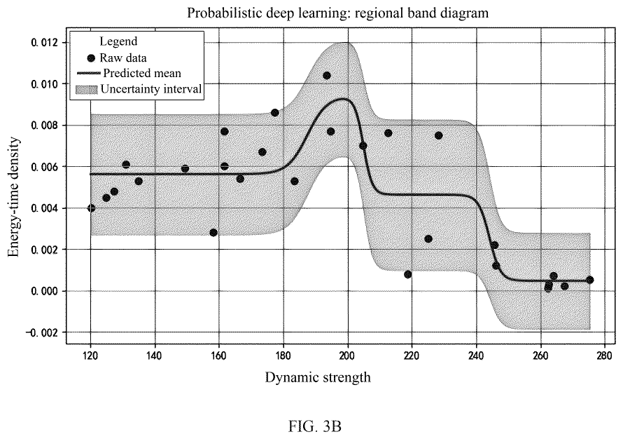

In one embodiment, a method of constructing the two-dimensional energy-time density map model based on the dynamic strength data and the energy-time density of the rocks, and by using the K-means clustering algorithm, the kernel density estimation or the interpolation model of the machine learning includes:
(a) based on energy-time density and dynamic strength data of the rocks under a same load, taking dynamic strength as an abscissa and a peak value of the energy-time density as an ordinate, drawing a basic energy-time density distribution scatter diagram, and calculating a category and density probability of each scatter based on the K-means clustering algorithm and the kernel density estimation to generate the two-dimensional energy-time density map model, as shown in FIG. 3A;
calculating a position of each energy-time density and each dynamic strength, and performing cluster analysis to obtain the density probability of each cluster; assuming that a data point set is D={(x₁,y₁), (x₂,y₂), . . . , (xₙ,yₙ)}, where x is the dynamic strength and y is the peak value of the energy-time density, and based on the K-means clustering algorithm, an objective function of a clustering process may be expressed as:

$$J = {{\sum }_{k = 1}}^{K} {{\sum }_{i = 1}}^{n} {‖ ( {x}_{i} , {y}_{i} ) - {c}_{k} ‖}^{2} ;$$

where J is the objective function, K is a number of clusters, cₖ is a center of a k-th cluster, and ∥(xᵢ, yᵢ)−cₖ∥ is a Euclidean distance between the data points and a cluster center;
using the kernel density estimation to estimate the probability density of the data points and generating a two-dimensional probability density map:

$$f ( x , y ) = \frac{1}{{nh}_{x} {h}_{y}} {\sum }_{i = 1}^{n} K ( \frac{x - {x}_{i}}{{h}_{x}} , \frac{y - {y}_{i}}{{h}_{y}} ) ;$$

where f(x,y) is a probability density function, n is a number of the data points, hₓ and h_{y} are bandwidth parameters, and K is a kernel function, usually a Gaussian kernel;
(b) based on energy-time density and dynamic strength data of each of the rocks under different loads, interpolating and extrapolating energy-time density data under different strain rates, and using the interpolation model of the machine learning to construct a continuous and smooth spatial distribution map of the energy-time density to obtain a two-dimensional energy-time density map distribution model, as shown in FIG. 3B;
using the interpolation model of the machine learning to interpolate the energy-time density under different strain rates:

$$\hat{y} = f ( x , θ ) = W \cdot x + b ;$$

where ŷ is a predicted energy-time density value, f(x,θ) is an output of an multiplayer perceptron (MLP) model, W is a weight matrix, b is a bias term, and x is an input feature.

Based on the same inventive concept, an embodiment of the present disclosure also provides a method for predicting an energy-time density map model for evaluating rock energy consumption.

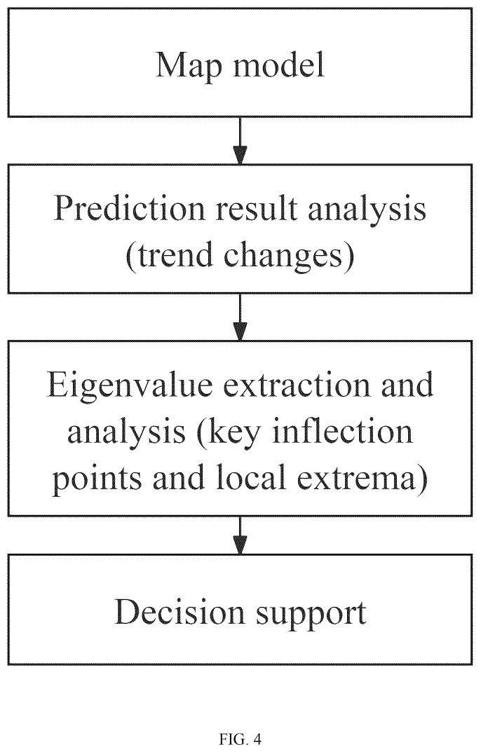

As shown in FIG. 4, FIG. 4 is a method for predicting an energy-time density map model for evaluating rock energy consumption provided by an embodiment of the present disclosure. A prediction method is to perform trend prediction based on the two-dimensional energy-time density map model to obtain an energy-time density prediction result.

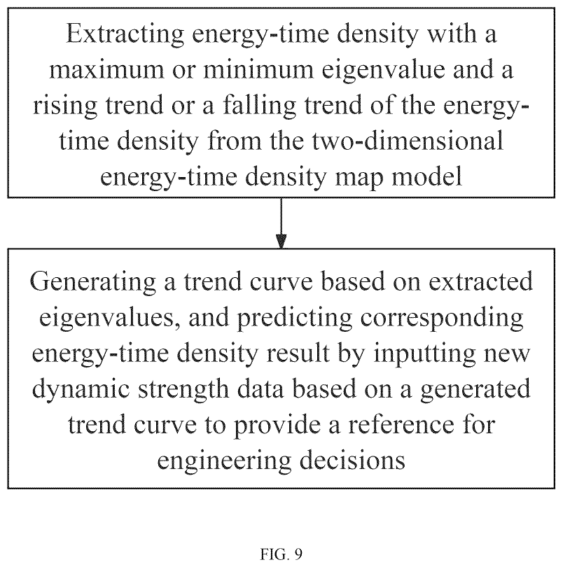

In one embodiment, As shown in FIG. 9, a method of performing the trend prediction based on the two-dimensional energy-time density map model to obtain the energy-time density prediction result includes:
extracting energy-time density with a maximum or minimum eigenvalue and a rising trend or a falling trend of the energy-time density from the two-dimensional energy-time density map model; and
generating a trend curve based on extracted eigenvalues, and predicting corresponding energy-time density result by inputting new dynamic strength data based on a generated trend curve to provide a reference for engineering decisions.

Based on the method for predicting an energy-time density map model for evaluating rock energy consumption provided by an embodiment of the present disclosure, this method is not only applicable to various rock types and different loading conditions, but also may predict energy dissipation through the two-dimensional energy-time density map model, providing a scientific basis for optimizing engineering decisions. In short, this method improves the accuracy, visualization effect, and applicability of rock energy consumption evaluation, and provides strong support for the optimization of blasting schemes, rock fracture control, and energy utilization.

### Embodiment 1

An energy-time density map for a typical metal mine is drawn. The mine is rich in various typical rocks, and the energy-time density map is drawn.

### Step 1. Data Collection and Processing

a) Collecting and processing various typical rock specimens into the specimen size required for SHPB tests;
b) Conducting SHPB tests on the rock samples to be tested, performing five tests for each typical rock, and collecting stress, strain signals, of the rocks under impact loads; and
c) Inputting the collected stress and strain time-series data into the deep learning denoising model, and automatically filtering high-frequency noise in the data through DAE to retain the main information related to rock stress characteristics, facilitating subsequent calculations.

### Step 2. Calculation of Dynamic Strength

Calculating the dynamic strength data of various rocks according to the one-dimensional stress wave theory of elastic rods and the three assumptions of SHPB tests. The calculation method is as follows:

$$\{ \begin{matrix}σ ( t ) = \frac{AE}{2 {A}_{s}} [ {ε}_{I} ( t ) + {ε}_{R} ( t ) + {ε}_{T} ( t ) ] \\ ε ( t ) = \frac{C}{{l}_{s}} {\int }_{0}^{t} [ {ε}_{I} ( t ) - {ε}_{R} ( t ) - {ε}_{T} ( t ) ] dt \\ \dot{ε} ( t ) = \frac{C}{{l}_{s}} [ {ε}_{I} ( t ) - {ε}_{R} ( t ) - {ε}_{T} ( t ) ]\end{matrix} ;$$

where A, E, and C are a cross-sectional area, elastic modulus, and longitudinal wave velocity of a pressure bar, respectively; Aₛ and lₛ are a cross-sectional area and a length of a specimen, respectively; and &I (t), ε_{R}(t), and ε_{T}(t) are incident strain, reflected strain, and transmitted strain of the pressure bar.

### Step 3. Calculation of Energy-Time Density

Using the three-wave method to calculate the energy-time density of typical rocks under the same load condition;
where calculation methods of each part of energy are as follows:

$${W}_{i} = \frac{{A}_{0}}{{ρ}_{0} {C}_{0}} {\int }_{0}^{t} {σ}_{i}^{2} ( t ) dt ;$$

$${W}_{r} = \frac{{A}_{0}}{{ρ}_{0} {C}_{0}} {\int }_{0}^{t} {σ}_{r}^{2} ( t ) dt ;$$

$${W}_{t} = \frac{{A}_{0}}{{ρ}_{0} {C}_{0}} {\int }_{0}^{t} {σ}_{t}^{2} ( t ) dt ; and$$

$${W}_{d} = {W}_{i} - {W}_{r} - {W}_{t} ;$$

where Wᵢ, Wᵣ, Wₜ, and W_{d} are incident energy, reflected energy, transmitted energy, and absorbed energy, respectively; σᵢ, σᵣ, and σₜ are incident stress, reflected stress, and transmitted stress, respectively; A₀ is a cross-sectional area of a rod; C₀ is elastic wave velocity in the pressure bar; ρ₀ is density of an elastic rod;
the energy consumption density is defined as dissipated energy per unit volume:

$${U}_{d} = \frac{{W}_{d}}{V} ;$$

where U_{d} is the energy consumption density, W_{d} is the absorbed energy, and V is a volume; and
the energy-time density of the rocks is calculated according to the energy consumption density; the energy-time density reflects dissipated energy of the rock per unit volume per unit time, and a calculation formula is as follows:

$${E}_{VT} = \frac{{U}_{d}}{T} ;$$

where E_{VT} is the energy-time density, U_{d} is the energy consumption density, and T is action time of a reflected wave.

### Step 4. Construction of Energy-Time Density Map

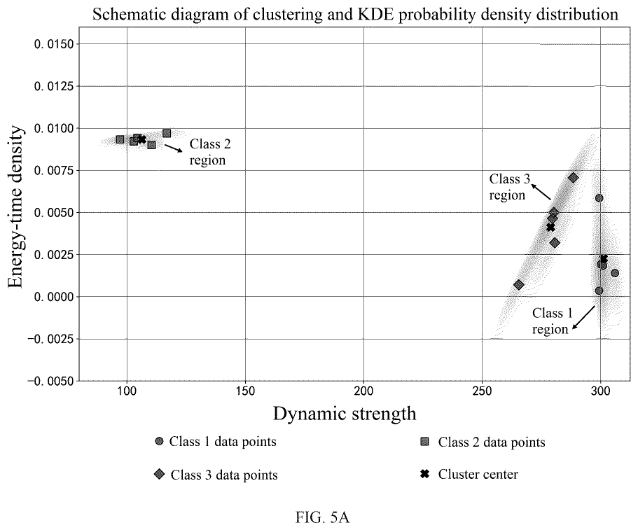

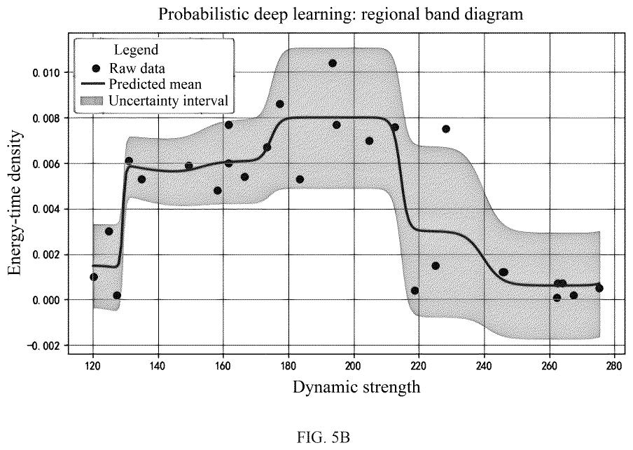

a) Two-dimensional map model of typical rocks: Based on the energy-time density and dynamic strength data of typical rocks under the same load, taking the dynamic strength as the abscissa and the peak value of energy-time density as the ordinate, drawing a basic energy-time density distribution scatter diagram, and calculating the category and density probability of each scatter based on the K-means clustering algorithm and KDE probability density algorithm to generate a two-dimensional energy-time density map model, as shown in FIG. 5A.
b) Two-dimensional map model of typical loads: Based on the energy-time density and dynamic strength data of a single rock under different loads, interpolating and extrapolating the energy-time density data under different test conditions (different strain rates), and using the interpolation model of machine learning (interpolation network based on deep learning) to construct a continuous and smooth spatial distribution map of energy-time density to obtain a more comprehensive two-dimensional energy-time density map distribution model, as shown in FIG. 5B.
c) Result prediction and model feature marking:
The generated map model may further generate its trend curve, which may then be used for relevant predictions. At the same time, statistical analysis methods may be combined to find its eigenvalues, providing a basis for subsequent decisions.

### Step 5. Map Analysis and Decision Evaluation

a) Quantitative analysis: Extracting eigenvalues from the map (maximum and minimum energy-time density, rising and falling trends);
b) Result prediction: According to the generated trend curve, relevant results may be predicted and analyzed. Inputting dynamic strength data may predict the energy-time density result, and the maximum energy-time density range may be found to further guide the production to use specific energy parameters for crushing and other work, providing a reference for engineering decisions.

### Embodiment 2

Based on the same inventive concept as Embodiment 1, the difference between Embodiment 2 of the present disclosure and Embodiment 1 is that the rock types calculated are different, and different rock types exhibit different rock dynamic characteristics and energy dissipation.

An energy-time density map for a typical limestone mine is drawn. The rock types in the mine are relatively single, and the energy-time density map is drawn.

### Step 1. Data Collection and Processing

a) Collecting and processing various typical rock specimens into the specimen size required for SHPB tests;
b) Conducting SHPB experiments on the rock samples to be tested, performing five tests for each typical rock, and collecting stress, strain signals of the rocks under impact loads;
c) Inputting the collected stress and strain time-series data into the deep learning denoising model, and automatically filtering high-frequency noise in the data through DAE to retain the main information related to rock stress characteristics, facilitating subsequent calculations.

### Step 2. Calculation of Dynamic Strength

Calculating the dynamic strength data of various rocks according to the one-dimensional stress wave theory of elastic rods and the three assumptions of SHPB tests. The calculation method is as follows:

$$\{ \begin{matrix}σ ( t ) = \frac{AE}{2 {A}_{s}} [ {ε}_{I} ( t ) + {ε}_{R} ( t ) + {ε}_{T} ( t ) ] \\ ε ( t ) = \frac{C}{{l}_{s}} {\int }_{0}^{t} [ {ε}_{I} ( t ) - {ε}_{R} ( t ) - {ε}_{T} ( t ) ] dt \\ \dot{ε} ( t ) = \frac{C}{{l}_{s}} [ {ε}_{I} ( t ) - {ε}_{R} ( t ) - {ε}_{T} ( t ) ]\end{matrix} ;$$

where A, E, and C are a cross-sectional area, elastic modulus, and longitudinal wave velocity of a pressure bar, respectively; Aₛ and lₛ are a cross-sectional area and a length of a specimen, respectively; and ε_{I}(t), ε_{R}(t), and ε_{T}(t) are incident strain, reflected strain, and transmitted strain of the pressure bar.

### Step 3. Calculation of Energy-Time Density

Using the three-wave method to calculate the energy-time density of typical rocks under the same load condition.

Calculation methods of each part of energy are as follows:

$${W}_{i} = \frac{{A}_{0}}{{ρ}_{0} {C}_{0}} {\int }_{0}^{t} {σ}_{i}^{2} ( t ) dt ;$$

$${W}_{r} = \frac{{A}_{0}}{{ρ}_{0} {C}_{0}} {\int }_{0}^{t} {σ}_{r}^{2} ( t ) dt ;$$

$${W}_{t} = \frac{{A}_{0}}{{ρ}_{0} {C}_{0}} {\int }_{0}^{t} {σ}_{t}^{2} ( t ) dt ; and$$

$${W}_{d} = {W}_{i} - {W}_{r} - {W}_{t} ;$$

where Wᵢ, Wᵣ, Wₜ, and W_{d} are incident energy, reflected energy, transmitted energy, and absorbed energy, respectively; σᵢ, σᵣ, and σₜ are incident stress, reflected stress, and transmitted stress, respectively; A₀ is a cross-sectional area of a rod; C₀ is elastic wave velocity in the pressure bar; ρ₀ is density of an elastic rod;
the energy consumption density is defined as dissipated energy per unit volume:

$${U}_{d} = \frac{{W}_{d}}{V} ;$$

where U_{d} is the energy consumption density, W_{d} is the absorbed energy, and V is a volume; and
the energy-time density of the rocks is calculated according to the energy consumption density; the energy-time density reflects dissipated energy of the rock per unit volume per unit time, and a calculation formula is as follows:

$${E}_{VT} = \frac{{U}_{d}}{T} ;$$

where E_{VT} is the energy-time density, U_{d} is the energy consumption density, and T is action time of a reflected wave.

### Step 4. Construction of Energy-Time Density Map

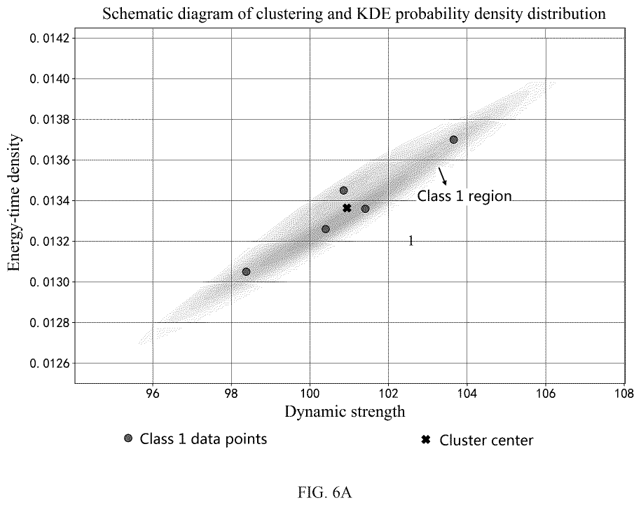

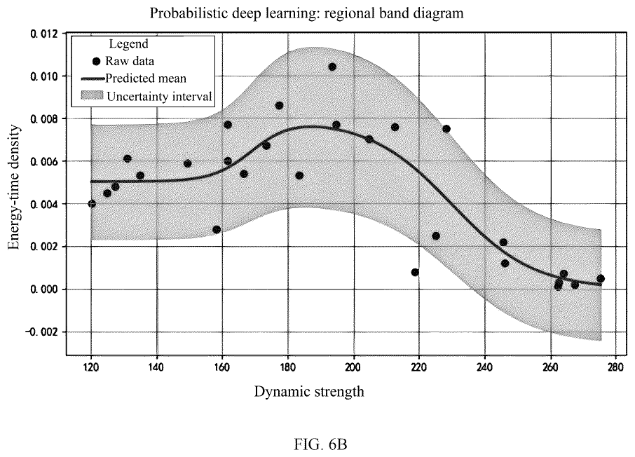

a) Two-dimensional map model of typical rocks: Based on the energy-time density and dynamic strength data of typical rocks under the same load, taking the dynamic strength as the abscissa and the peak value of energy-time density as the ordinate, drawing a basic energy-time density distribution scatter diagram, and calculating the category and density probability of each scatter based on the K-means clustering algorithm and KDE probability density algorithm to generate a two-dimensional energy-time density map model, as shown in FIG. 6A.
b) Two-dimensional map model of typical loads: Based on the energy-time density and dynamic strength data of a single rock under different loads, interpolating and extrapolating the energy-time density data under different test conditions (different strain rates), and use the interpolation model of machine learning (interpolation network based on deep learning) to construct a continuous and smooth spatial distribution map of energy-time density to obtain a more comprehensive two-dimensional energy-time density map distribution model, as shown in FIG. 6B.
c) Result prediction and model feature marking:

The generated map model may further generate its trend curve, which may then be used for relevant predictions. At the same time, statistical analysis methods may be combined to find its eigenvalues, providing a basis for subsequent decisions.

### Step 5. Map Analysis and Decision Evaluation

a) Quantitative analysis: Extracting eigenvalues from the map (maximum and minimum energy-time density, rising and falling trends).
b) Result prediction: According to the generated trend curve, relevant results may be predicted and analyzed. Inputting dynamic strength data may predict the energy-time density result, and the maximum energy-time density range may be found to further guide the production to use specific energy parameters for crushing and other work, providing a reference for engineering decisions.

The above describes an embodiment of the present disclosure in detail, but the content is only an optional embodiment of the present disclosure and shall not be considered as limiting the scope of implementation of the present disclosure. All equivalent changes and improvements made according to the scope of the present disclosure shall still fall within the scope of the patent of the present disclosure.

What is claimed is:

1. A method for establishing an energy-time density map model for evaluating rock energy consumption, wherein the method for establishing the energy-time density map model comprises following steps:
selecting rock samples, and conducting Hopkinson experiments to obtain stress and strain signals;
processing obtained stress-strain signals by using a deep learning denoising model to obtain purified stress-strain signals;
calculating dynamic strength data and energy-time density based on the purified stress-strain signals; and
constructing a two-dimensional energy-time density map model based on the dynamic strength data and the energy-time density of rocks, and by using a K-means clustering algorithm, kernel density estimation or an interpolation model of machine learning;
wherein a method of constructing the two-dimensional energy-time density map model based on the dynamic strength data and the energy-time density of the rocks, and by using the K-means clustering algorithm, the kernel density estimation or the interpolation model of the machine learning comprises:
(a) based on energy-time density and dynamic strength data of the rocks under a same load, taking dynamic strength as an abscissa and a peak value of the energy-time density as an ordinate, drawing a basic energy-time density distribution scatter diagram, and calculating a category and density probability of each scatter based on the K-means clustering algorithm and the kernel density estimation to generate the two-dimensional energy-time density map model;
calculating a position of each energy-time density and each dynamic strength, and performing cluster analysis to obtain the density probability of each cluster; assuming a data point set is D={(x₁,y₁), (x₂,y₂), . . . , (xₙ,yₙ)}, wherein x is the dynamic strength and y is the peak value of the energy-time density, and based on the K-means clustering algorithm, an objective function of a clustering process may be expressed as:
$$J = {\sum }_{k = 1}^{K} {\sum }_{i = 1}^{n} {‖ ( {x}_{i} , {y}_{i} ) - {c}_{k} ‖}^{2} ;$$
wherein J is the objective function, K is a number of clusters, cₖ is a center of a k-th cluster, and ∥(xᵢ, yᵢ)−cₖ is a Euclidean distance between data points and a cluster center;
using the kernel density estimation to estimate probability density of the data points, and generating a two-dimensional probability density map:
$$f ( x , y ) = \frac{1}{{nh}_{x} {h}_{y}} {\sum }_{i = 1}^{n} K ( \frac{x - {x}_{i}}{{h}_{x}} , \frac{y - {y}_{i}}{{h}_{y}} ) ;$$
wherein f(x,y) is a probability density function, n is a number of data points, hₓ and h_{y} are bandwidth parameters, and K is a kernel function, usually a Gaussian kernel;
(b) based on energy-time density and dynamic strength data of each of the rocks under different loads, interpolating and extrapolating energy-time density data under different strain rates, and using the interpolation model of the machine learning to construct a continuous and smooth spatial distribution map of the energy-time density to obtain a two-dimensional energy-time density map distribution model;
using the interpolation model of the machine learning to interpolate the energy-time density under different strain rates:
$$\hat{y} = f ( x , θ ) = W \cdot x + b ;$$
wherein ŷ is a predicted energy-time density value, f(x,θ) is an output of an multiplayer perceptron (MLP) model, W is a weight matrix, b is a bias term, and x is an input feature.

2. The method for establishing the energy-time density map model for evaluating the rock energy consumption according to claim 1, wherein a method of selecting the rock samples and conducting the Hopkinson experiments to obtain the stress and the strain signals comprises:
collecting and processing a plurality of types of the rock samples into specimen sizes required for the Hopkinson experiments; and
conducting the Hopkinson experiments on the rock samples to be tested, performing five experiments for each type of the rock samples, and collecting stress and strain signals of the rocks under impact loads.

3. The method for establishing the energy-time density map model for evaluating the rock energy consumption according to claim 1, wherein a method of processing the obtained stress-strain signals by using the deep learning denoising model comprises:
inputting collected stress and strain signal data into the deep learning denoising model, and automatically filtering high-frequency noise in data through a denoising autoencoder (DAE) to obtain the purified stress-strain signals.

4. The method for establishing the energy-time density map model for evaluating the rock energy consumption according to claim 1, wherein steps for obtaining the dynamic strength data comprise:
a calculation method of calculating the dynamic strength data of various rocks based on the purified stress-strain signals and combined with one-dimensional stress wave theory of elastic rods is as follows:
$$\{ \begin{matrix}σ ( t ) = \frac{AE}{2 {A}_{s}} [ {ε}_{I} ( t ) + {ε}_{R} ( t ) + {ε}_{T} ( t ) ] \\ ε ( t ) = \frac{C}{{l}_{s}} {\int }_{0}^{t} [ {ε}_{I} ( t ) - {ε}_{R} ( t ) - {ε}_{T} ( t ) ] dt \\ \dot{ε} ( t ) = \frac{C}{{l}_{s}} [ {ε}_{I} ( t ) - {ε}_{R} ( t ) - {ε}_{T} ( t ) ]\end{matrix} ;$$
wherein A, E, and C are a cross-sectional area, elastic modulus, and longitudinal wave velocity of a pressure bar, respectively; Aₛ and lₛ are a cross-sectional area and a length of a specimen, respectively; and ε_{I}(t), ε_{R}(t), and ε_{T}(t) are incident strain, reflected strain, and transmitted strain of the pressure bar.

5. The method for establishing the energy-time density map model for evaluating the rock energy consumption according to claim 1, wherein steps for obtaining the energy-time density comprise:
calculating the energy-time density under a same load condition by using a three-wave method based on the purified stress-strain signals;
wherein calculation methods of each part of energy are as follows:
$${W}_{i} = \frac{{A}_{0}}{{ρ}_{0} {C}_{0}} {\int }_{0}^{t} {σ}_{i}^{2} ( t ) dt ;$$
$${W}_{r} = \frac{{A}_{0}}{{ρ}_{0} {C}_{0}} {\int }_{0}^{t} {σ}_{r}^{2} ( t ) dt ;$$
$${W}_{t} = \frac{{A}_{0}}{{ρ}_{0} {C}_{0}} {\int }_{0}^{t} {σ}_{t}^{2} ( t ) dt ; and$$
$${W}_{d} = {W}_{i} - {W}_{r} - {W}_{t} ;$$
wherein Wᵢ, Wᵣ, Wₜ, and W_{d} are incident energy, reflected energy, transmitted energy, and absorbed energy, respectively; σᵢ, σᵣ, and σₜ are incident stress, reflected stress, and transmitted stress, respectively; A₀ is a cross-sectional area of a rod; C₀ is elastic wave velocity in the pressure bar; ρ₀ is density of an elastic rod;
energy consumption density is defined as dissipated energy per unit volume:
$${U}_{d} = \frac{{W}_{d}}{V} ;$$
wherein U_{d} is the energy consumption density, W_{d} is the absorbed energy, and V is a volume; and
the energy-time density of the rocks is calculated according to the energy consumption density; the energy-time density reflects dissipated energy of the rock per unit volume per unit time, and a calculation formula is as follows:
$${E}_{VT} = \frac{{U}_{d}}{T} ;$$
wherein E_{VT} is the energy-time density, U_{d} is the energy consumption density, and T is action time of a reflected wave.

6. A method for predicting an energy-time density map model for evaluating rock energy consumption, wherein an establishment of the energy-time density map model is based on the method for establishing the energy-time density map model for evaluating the rock energy consumption according to claim 1, and a prediction method is to perform trend prediction based on the two-dimensional energy-time density map model to obtain an energy-time density prediction result.

7. The method for predicting the energy-time density map model for evaluating the rock energy consumption according to claim 6, wherein a method of performing the trend prediction based on the two-dimensional energy-time density map model to obtain the energy-time density prediction result comprises:
extracting energy-time density with a maximum or minimum eigenvalue and a rising trend or a falling trend of the energy-time density from the two-dimensional energy-time density map model; and
generating a trend curve based on extracted eigenvalues, and predicting corresponding energy-time density result by inputting new dynamic strength data based on a generated trend curve to provide a reference for engineering decisions.
